(19) United States
(12) Patent Application Publication
(10) Pub. No.: US 2024/0225683 A9
(43) Pub. Date: Jul. 11, 2024

(54) ARTICULATING ULTRASONIC SURGICAL INSTRUMENTS HAVING DISTALLY POSITIONED TRANSDUCERS

(71) Applicant: Covidien LP, Mansfield, MA (US)

(72) Inventors: Matthew S. Cowley, Frederick, CO (US); James R. Fagan, Erie, CO (US); Adam J. Neil, Provo, UT (US); Grant J. Hamilton, Highlands Ranch, CO (US); Jason C. Bledsoe, Alpin, UT (US); Nolan H. Howes, West Haven, UT (US); Wesley S. Bohn, Ogden, UT (US)

(21) Appl. No.: 17/970,257

(22) Filed: Oct. 20, 2022

(51) Int. Cl. A61B 17/32

(52) U.S. Cl. CPC A61B 17/320092; A61B 2017/2932; A61B 2017/320071; A61B 2017/320074

(57) ABSTRACT

An articulating surgical instrument includes a housing, a shaft extending distally from the housing, an end effector assembly, and an articulating component interconnecting the shaft and end effector assembly. The articulating component includes a proximal disk connected to the shaft, a distal disk connected to the end effector assembly, an intermediate disk, a first flexible interconnect connecting the proximal and intermediate disks, and a second flexible interconnect connecting the intermediate and distal disks. The first flexible interconnect defines a single plane of bending to enable articulation of the end effector assembly relative to the shaft within a first plane. The second flexible interconnect defines a single plane of bending substantially perpendicular to the single plane of bending of the first flexible interconnect to enable articulation of the end effector assembly relative to the shaft within a second plane substantially perpendicular to the first plane.

### FIELD

This disclosure relates to surgical instruments and systems and, more particularly, to articulating ultrasonic surgical instruments having distally positioned transducers such as for use in surgical robotic systems.

### BACKGROUND

Ultrasonic surgical instruments and systems utilize ultrasonic energy, i.e., ultrasonic vibrations, to treat tissue. More specifically, a typical ultrasonic surgical instrument or system includes a transducer configured to produce mechanical vibration energy at ultrasonic frequencies that is transmitted along a waveguide to an ultrasonic end effector configured to treat, e.g., seal and/or transect, tissue.

Some ultrasonic surgical instruments and systems incorporate rotation features, thus enabling rotation of the end effector to a desired orientation within a surgical site. However, the ability to manipulate an end effector within the surgical site via rotation alone is limited.

Adding articulation capability to an ultrasonic surgical instrument increases the positions and orientations the end effector can achieve within a surgical site. However, with the addition of articulation capability comes the challenges of routing mechanical actuators, power signals, control signals, and/or mechanical vibration energy to the end effector.

### SUMMARY

As used herein, the term “distal” refers to the portion that is being described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. Further, to the extent consistent, any of the aspects described herein may be used in conjunction with any or all of the other aspects described herein.

Provided in accordance with aspects of this disclosure is an articulating surgical instrument including a housing, a shaft extending distally from the housing, an end effector assembly, and an articulating component interconnecting the shaft and end effector assembly. The articulating component includes a proximal disk connected to the shaft, a distal disk connected to the end effector assembly, an intermediate disk, a first flexible interconnect connecting the proximal and intermediate disks, and a second flexible interconnect connecting the intermediate and distal disks. The first flexible interconnect defines a single plane of bending to enable articulation of the end effector assembly relative to the shaft within a first plane. The second flexible interconnect defines a single plane of bending substantially perpendicular to the single plane of bending of the first flexible interconnect to enable articulation of the end effector assembly relative to the shaft within a second plane substantially perpendicular to the first plane.

In an aspect of this disclosure, the articulating component is a monolithic, single piece of material.

In an aspect of this disclosure, each of the first and second flexible interconnects defines a beam configuration having a pair of opposed relatively narrow sides and a pair of opposed relatively broad sides. In such aspects, each of the first and second flexible interconnects may be aligned relative to a longitudinal axis defined through the shaft.

In another aspect of this disclosure, a first pair of opposed articulation cables extends from the shaft through the proximal and intermediate disks. The first pair of opposed articulation cables is anchored distally of the intermediate disk and configured to move in opposite directions to articulate the end effector assembly relative to the shaft within the first plane.

In yet another aspect of this disclosure, a second pair of opposed articulation cables extends from the shaft through the proximal, intermediate, and distal disks. The second pair of opposed articulation cables is anchored distally of the distal disk and configured to move in opposite directions to articulate the end effector assembly relative to the shaft within the second plane. In aspects, the first and second pairs of opposed articulation cables are offset about 90 degrees relative to one another.

In still another aspect of this disclosure, the end effector assembly includes a body, an ultrasonic transducer housed within the body, and an ultrasonic blade extending distally from the body and configured to treat tissue with ultrasonic energy produced by the ultrasonic transducer.

In still yet another aspect of this disclosure, the end effector assembly further includes a jaw member pivotable relative to the ultrasonic blade between an open position and a closed position for clamping tissue therebetween. In such aspects, at least one jaw actuation cable may be routed through the articulating component to the end effector assembly.

In another aspect of this disclosure, the distal disk is connected to the end effector assembly by a releasable engagement. The releasable engagement may include a first connector and a second connector. The second connector includes first and second rails defining a slot therebetween and an engagement tab positioned towards an open end of the slot. The first connector is transversely slidable between the first and second rails and into the slot. The engagement tab is configured to releasably engage the first connector within the slot upon sufficient transverse sliding of the first connector into the slot.

Another articulating surgical instrument provided in accordance with aspects of this disclosure includes a housing, a shaft assembly extending distally from the housing and including a proximal shaft and a distal articulating section, and an end effector assembly releasably coupled to the distal articulating section of the shaft assembly by a releasable engagement. The releasable engagement includes a first connector disposed on one of the end effector assembly or the distal articulating section and a second connector disposed on another of the end effector assembly or the distal articulating section. The second connector includes first and second rails defining a slot therebetween and an engagement tab positioned towards an open end of the slot. The first connector is transversely slidable between the first and second rails and into the slot. The engagement tab is configured to releasably engage the first connector within the slot upon sufficient transverse sliding of the first connector into the slot.

In an aspect of this disclosure, the first connector is disposed on the end effector assembly and the second connector is disposed on the distal articulating section.

In another aspect of this disclosure, the second connector further includes a living hinge having the engagement tab disposed at a free end of the living hinge.

In still another aspect of this disclosure, the end effector assembly includes a body having the first or second connector extending therefrom, an ultrasonic transducer housed within the body, and an ultrasonic blade extending distally from the body and configured to treat tissue with ultrasonic energy produced by the ultrasonic transducer.

In yet another aspect of this disclosure, the end effector assembly further includes a jaw member pivotable relative to the ultrasonic blade between an open position and a closed position for clamping tissue therebetween.

A rotating and articulating surgical instrument provided in accordance with this disclosure includes a housing, a shaft assembly extending distally from the housing and including a proximal shaft and a distal articulating section, an end effector assembly coupled to the distal articulating section and configured to articulate relative to the proximal shaft within at least one plane, and a rotation mechanism including a cable extending through the distal articulating section of the shaft assembly to the end effector assembly. The cable includes a first portion, a second portion, and a partially looped portion interconnecting the first and second portions. The partially looped portion is disposed at least partially within an annular track of a proximal head of the end effector assembly. Movement of the first and second portions of the cable in opposite directions slides the partially looped portion through the annular track to generate torque to thereby rotate the end effector assembly relative to the shaft assembly.

In an aspect of this disclosure, movement of the first and second portions of the cable in opposite directions in a first manner slides the partially looped portion through the annular track in a clockwise direction to generate torque to thereby rotate the end effector assembly relative to the shaft assembly in the clockwise direction, while movement of the first and second portions of the cable in opposite directions in a second, opposite manner slides the partially looped portion through the annular track in a counterclockwise direction to generate torque to thereby rotate the end effector assembly relative to the shaft assembly in the counterclockwise direction.

In another aspect of this disclosure, movement of the first and second portions of the cable in opposite directions slides the partially looped portion through the annular track and, as a result of friction between the partially looped portion of the cable and the annular track, torque is generated to thereby rotate the end effector assembly relative to the shaft assembly.

The details of one or more aspects of this disclosure are set forth in the accompanying drawings and the description below. Other features, objects, and advantages of the techniques described in this disclosure will be apparent from the description and drawings, and from the claims.

### BRIEF DESCRIPTION OF DRAWINGS

Various aspects and features of this disclosure are described hereinbelow with reference to the drawings wherein like numerals designate identical or corresponding elements in each of the several views.

FIG. 1 is a schematic illustration of a surgical robotic system including a control tower, a console, and one or more surgical robotic arms according to aspects of this disclosure;

FIG. 2 is a perspective view of a surgical robotic arm of the surgical robotic system of FIG. 1 according to aspects of this disclosure;

FIG. 3 is a perspective view of a setup arm with the surgical robotic arm of the surgical robotic system of FIG. 1 according to aspects of this disclosure;

FIG. 4 is a schematic diagram of a computer architecture of the surgical robotic system of FIG. 1 according to aspects of this disclosure;

FIG. 5 is a perspective view of a proximal portion of an ultrasonic surgical instrument provided in accordance with this disclosure and configured for mounting on a robotic arm of a surgical robotic system such as the surgical robotic system of FIG. 1;

FIGS. 6A and 6B are representative, perspective views of a distal portion of the ultrasonic surgical instrument of FIG. 5 disposed in aligned and articulated positions, respectively;

FIG. 7 is a perspective view of the proximal portion of the surgical instrument of FIG. 5 with portions removed to illustrate internal operating features therein;

FIG. 8 is a detailed, front perspective view of the distal portion of the ultrasonic surgical instrument of FIG. 5;

FIG. 9 is an enlarged, perspective view of the area of detail indicated as “9” in FIG. 8;

FIGS. 10A and 10B are perspective and side views, respectively, of an articulating component of the ultrasonic surgical instrument of FIG. 5;

FIG. 11 is side view of the end effector assembly of the ultrasonic surgical instrument of FIG. 5;

FIG. 12 is a perspective view of the end effector assembly of the ultrasonic surgical instrument of FIG. 5;

FIG. 13 is an exploded, perspective view of a jaw member of the end effector assembly of FIG. 11 and a jaw actuation cable of the ultrasonic surgical instrument of FIG. 5;

FIG. 14 is a top view of a distal portion of the ultrasonic surgical instrument of FIG. 5;

FIG. 15A is a partial longitudinal, cross-sectional view illustrating the end effector assembly of FIG. 11 disengaged from the articulating component of the ultrasonic surgical instrument of FIG. 5;

FIG. 15B is an enlarged, partial longitudinal, cross-sectional view illustrating the engagement between the end effector assembly of FIG. 11 and the articulating component of the ultrasonic surgical instrument of FIG. 5;

FIG. 16 is a top, partial phantom view of a portion of another ultrasonic surgical instrument provided in accordance with this disclosure configured for mounting on a robotic arm of a surgical robotic system such as the surgical robotic system of FIG. 1, including a rotation mechanism coupling the articulating component with the end effector assembly;

FIG. 17 is perspective, partial longitudinal, cross-sectional view of a portion of the ultrasonic surgical instrument of FIG. 16 illustrating the rotation mechanism thereof; and

FIG. 18 is a perspective view of a distal portion of the ultrasonic surgical instrument of FIG. 16 with portions removed to better illustrate the rotation mechanism thereof.

### DETAILED DESCRIPTION

This disclosure provides articulating ultrasonic surgical instruments having distally positioned transducers. As described in detail below, the articulating ultrasonic surgical instruments of this disclosure may be configured for use with a surgical robotic system, which may include, for example, a surgical console, a control tower, and one or more movable carts having a surgical robotic arm coupled to a setup arm. The surgical console receives user inputs through one or more interface devices, which are interpreted by the control tower as movement commands for moving the surgical robotic arm. The surgical robotic arm includes a controller, which is configured to process the movement commands and to generate a torque command for activating one or more actuators of the robotic arm, which, in turn, move the robotic arm in response to the movement commands. Although described hereinbelow in connection with surgical robotic systems, the aspects and features of this disclosure may also be adapted for use with handheld articulating ultrasonic surgical instruments such as, for example, articulating endoscopic ultrasonic surgical instruments and/or articulating open ultrasonic surgical instruments.

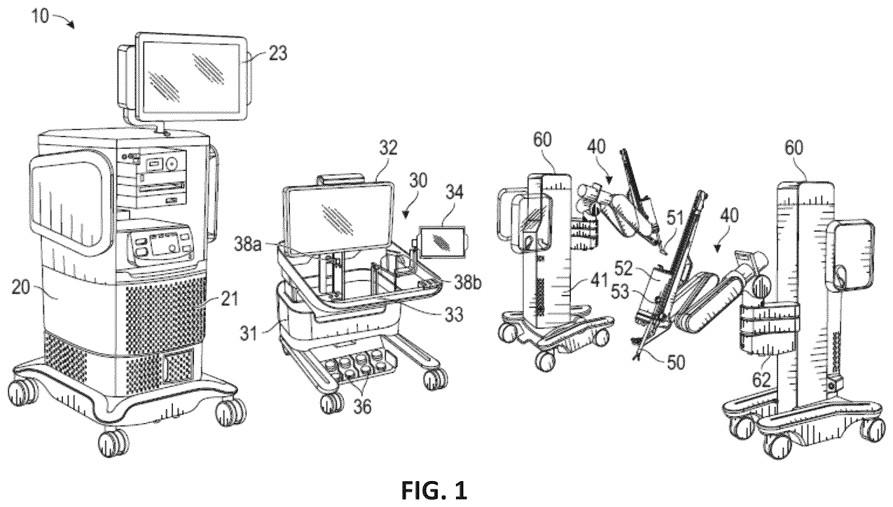

With reference to FIG. 1, a surgical robotic system 10 includes a control tower 20, which is connected to components of the surgical robotic system 10 including a surgical console 30 and one or more robotic arms 40. Each of the robotic arms 40 includes a surgical instrument 50 removably coupled thereto. Each of the robotic arms 40 is also coupled to a movable cart 60.

The one or more surgical instruments 50 may be configured for use during minimally invasive surgical procedures and/or open surgical procedures. In aspects, one of the surgical instruments 50 may be an endoscope, such as an endoscopic camera 51, configured to provide a video feed for the clinician. In further aspects, one of the surgical instruments 50 may be an energy based surgical instrument such as, for example, an electrosurgical forceps or ultrasonic sealing and dissection instrument configured to seal tissue by grasping tissue between opposing structures and applying electrosurgical energy or ultrasonic energy, respectively, thereto. In yet further aspects, one of the surgical instruments 50 may be a surgical stapler including a pair of jaws configured to clamp tissue, deploy a plurality of tissue fasteners, e.g., staples, through the clamped tissue, and/or to cut the stapled tissue. In aspects, one of the surgical instruments 50 is an articulating ultrasonic surgical instrument having a distally positioned transducer in accordance with this disclosure and as described in greater detail below.

Endoscopic camera 51, as noted above, may be configured to capture video of the surgical site. In such aspects, the surgical console 30 includes a first display 32, which displays a video feed of the surgical site provided by endoscopic camera 51, and a second display 34, which displays a user interface for controlling the surgical robotic system 10. The first and second displays 32 and 34 may be touchscreen graphical user interface (GUI) displays allowing for receipt of various user inputs.

The surgical console 30 also includes a plurality of user interface devices, such as foot pedals 36 and a pair of handle controllers 38a and 38b which are used by a clinician to remotely control robotic arms 40. The surgical console further includes an armrest 33 used to support clinician's arms while operating the handle controllers 38a and 38b.

The control tower 20 includes a display 23, which may be a touchscreen GUI, and provides outputs to the various GUIs. The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and/or the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by this disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth® (an open wireless protocol for exchanging data over short distances, using short length radio waves, from fixed and mobile devices, creating personal area networks (PANs)), and/or ZigBee® (a specification for a suite of high level communication protocols using small, low-power digital radios based on the IEEE 122.15.4-2003 standard for wireless personal area networks (WPANs)).

The computers 21, 31, 41 may include any suitable processor(s) operably connected to a memory, which may include one or more of volatile, non-volatile, magnetic, optical, quantum, and/or electrical media, such as read-only memory (ROM), random access memory (RAM), electrically-erasable programmable ROM (EEPROM), non-volatile RAM (NVRAM), or flash memory. The processor(s) may be any suitable processor(s) (e.g., control circuit(s)) adapted to perform operations, calculations, and/or set of instructions including, but not limited to, a hardware processor, a field programmable gate array (FPGA), a digital signal processor (DSP), a central processing unit (CPU), a microprocessor, a quantum processor, and combinations thereof. Those skilled in the art will appreciate that the processor may be substituted for by using any logic processor (e.g., control circuit) adapted to execute algorithms, calculations, and/or set of instructions.

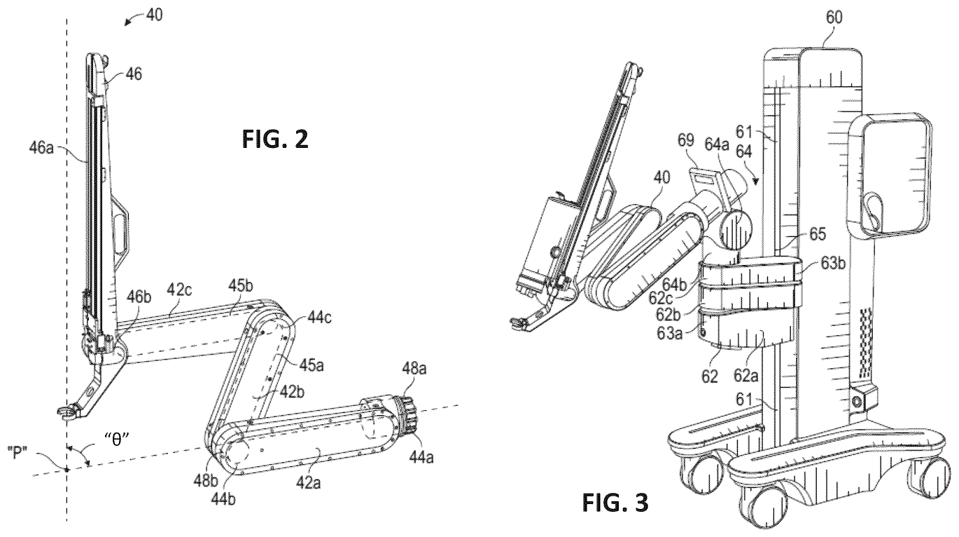

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40. The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In aspects, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

With reference again to FIG. 2, the robotic arm 40 also includes a holder 46 defining a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effectors) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c.

The robotic arm 40 further includes a plurality of manual override buttons 53 disposed on the IDU 52 and the setup arm 62, which may be used in a manual mode. For example, the clinician may press one of the buttons 53 to move the component associated with that button 53.

The joints 44a and 44b include an actuator 48a and 48b configured to drive the joints 44a, 44b, 44c relative to each other through a series of belts 45a and 45b or other mechanical linkages such as drive rods, cables, levers, and/or the like. In particular, the actuator 48a is configured to rotate the robotic arm 40 about a longitudinal axis defined by the link 42a.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b such that the actuator 48b is configured to rotate each of the links 42b, 42c and the holder 46 relative to one another. More specifically, links 42b, 42c and the holder 46 are passively coupled to the actuator 48b which enforces rotation about a remote center point “P” that lies at an intersection of the first axis defined by the link 42a and the second axis defined by the holder 46. Thus, the actuator 48b controls the angle “0” between the first and second axes allowing for orientation of the surgical instrument 50. Due to the interlinking of the links 42a, 42b, 42c and the holder 46 via the belts 45a and 45b, the angles between the links 42a, 42b, 42c and the holder 46 are also adjusted in order to achieve the desired angle “0.” In aspects, some or all of the joints 44a, 44b, 44c may include an actuator to obviate the need for mechanical linkages.

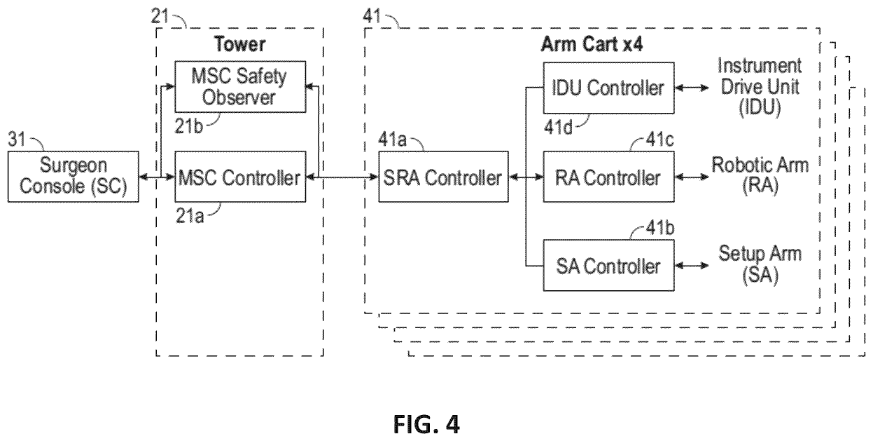

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and/or other inputs. The controller 21a processes these input positions to determine desired drive commands for each joint of the robotic arm 40 and/or the IDU 52 and communicates these to the computer 41 of the robotic arm 40. The controller 21a also receives the actual joint angles and uses this information to determine force feedback commands that are transmitted back to the computer 31 of the surgical console 30 to provide haptic or other feedback through the handle controllers 38a and 38b. The handle controllers 38a and 38b include one or more haptic feedback vibratory devices that output haptic feedback although visual, audible, and/or other feedback is also contemplated. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an IDU controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a communicates the actual joint angles back to the controller 21a.

The setup arm controller 41b controls each of joints 63a and 63b and the rotatable base 64 of the setup arm 62 and calculates desired motor movement commands (e.g., motor torque) for the pitch axis. The setup arm controller 41b also controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

With respect to control of the robotic arm 40, initially, a pose of the handle controller controlling the robotic arm 40, e.g., the handle controller 38a, is transformed into a desired pose of the robotic arm 40 through a hand eye transform function executed by the controller 21a. The hand eye function is embodied in software executable by the controller 21a or any other suitable controller of the surgical robotic system 10. The pose of the handle controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In aspects, the coordinate position is scaled down and the orientation is scaled up by the scaling function. In addition, the controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limiting mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

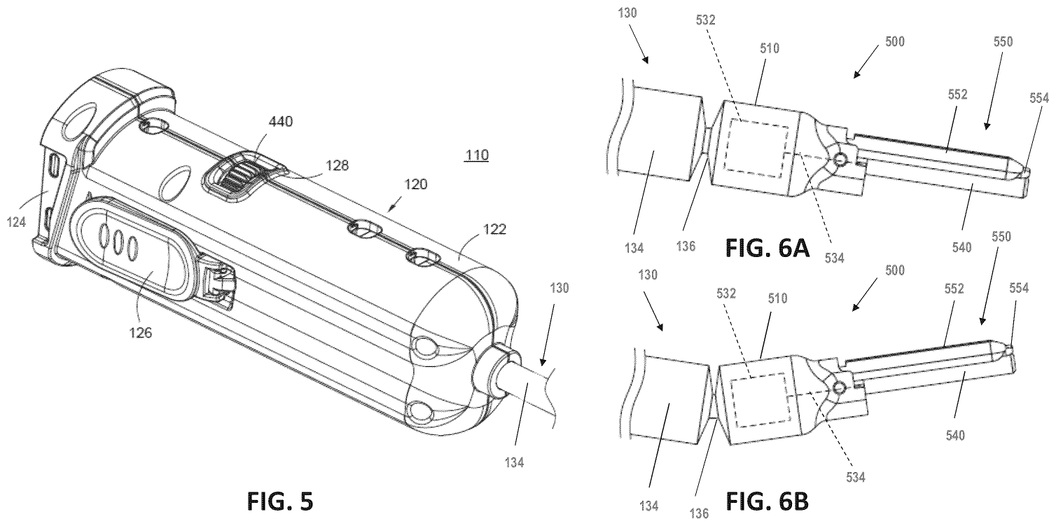

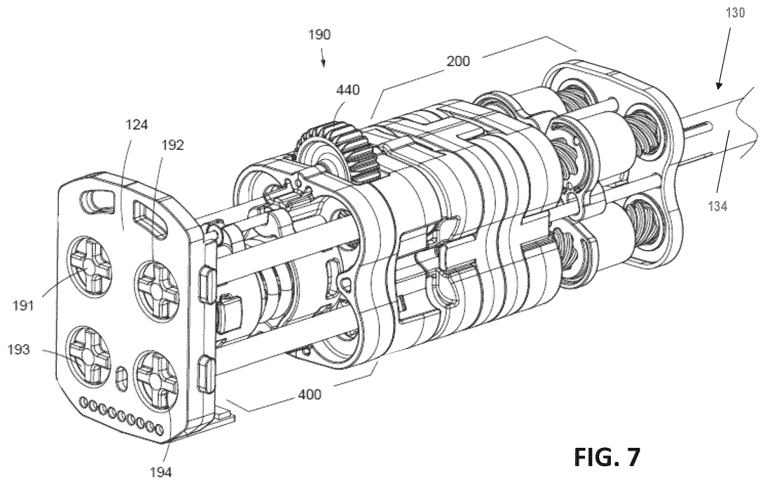

Turning to FIGS. 5-7, a surgical instrument 110 provided in accordance with this disclosure generally includes a housing 120, a shaft assembly 130 extending distally from housing 120, an end effector assembly 500 extending distally from shaft assembly 130, and an actuation assembly 190 (FIG. 7) disposed within housing 120 and operably associated with end effector assembly 500. Instrument 110 is detailed herein as an articulating ultrasonic surgical instrument configured for use with a surgical robotic system, e.g., surgical robotic system 10 (FIG. 1). However, the aspects and features of instrument 110 provided in accordance with this disclosure, as detailed below, are equally applicable for use with other suitable surgical instruments and/or in other suitable surgical systems, e.g., motorized, other power-driven systems, and/or manually actuated surgical systems (including handheld instruments).

Housing 120 of instrument 110 includes a body 122 and a proximal face plate 124 that cooperate to enclose actuation assembly 190 therein. Proximal face plate 124 includes through holes defined therein through which input couplers 191-194 of actuation assembly 190 extend. A pair of latch levers 126 (only one of which is illustrated in FIG. 5) extend outwardly from opposing sides of housing 120 to enable releasable engagement of housing 120 with a robotic arm of a surgical robotic system, e.g., robotic arm 40 of surgical robotic system 10 (FIG. 1). A window 128 defined through body 122 of housing 120 permits thumbwheel 440 to extend therethrough to enable manual manipulation of thumbwheel 440 from the exterior of housing 120 to permit manual opening and closing of end effector assembly 500.

Shaft assembly 130 of instrument 110 includes a proximal shaft 134 and an articulating section 136 disposed between and interconnecting proximal section 134 with end effector assembly 500. Articulating section 136 includes one or more articulating components such as, for example, one or more links, pivots, joints, flexible bodies, etc. A plurality of articulation cables 138 (FIG. 9) or other suitable actuators extend through articulating section 136. More specifically, articulation cables 138 (FIG. 9) may be operably coupled to end effector assembly 500 at the distal ends thereof and extend proximally through articulating section 136 of shaft assembly 130 and proximal shaft 134 of shaft assembly 130, and into housing 120, wherein articulation cables 138 (FIG. 9) operably couple with an articulation sub-assembly 200 of actuation assembly 190 to enable selective articulation of end effector assembly 500 relative to proximal shaft 134 and housing 120, e.g., about at least two axes of articulation (yaw and pitch articulation, for example).

With particular reference to FIGS. 6A and 6B, end effector assembly 500 includes a body 510 retaining an ultrasonic transducer 532 therein, an ultrasonic blade 540 operably coupled to ultrasonic transducer 532 via an ultrasonic horn 534 and extending distally from body 510, and a jaw member 550 operably coupled to body 510 to enable pivoting of jaw member 550 relative to ultrasonic blade 540 from a spaced-apart position to an approximated position to clamp tissue between ultrasonic blade 540 and jaw member 550. Jaw member 550 includes a rigid structural frame 552 that is operably coupled to body 510, and a compliant jaw liner 554 that is captured by rigid structural frame 552 and positioned to oppose ultrasonic blade 540 to enable clamping of tissue therebetween.

Referring again to FIGS. 5-7, actuation assembly 190 is disposed within housing 120 and includes an articulation sub-assembly 200 and a jaw drive sub-assembly 400. Articulation sub-assembly 200 is operably coupled between first and second input couplers 191, 192, respectively, of actuation assembly 190 and articulation cables 138 (FIG. 9) such that, upon receipt of appropriate inputs into first and/or second input couplers 191, 192, articulation sub-assembly 200 manipulates articulation cables 138 (FIG. 9) to articulate end effector assembly 500 in a desired direction, e.g., to pitch and/or yaw end effector assembly 500.

Jaw drive sub-assembly 400 operably couples fourth input coupler 194 of actuation assembly 190 with jaw member 550 such that, upon receipt of appropriate input, e.g., in a first rotational direction, into fourth coupler 194, jaw drive sub-assembly 400 pivots jaw member 550 towards the approximated position to clamp tissue between and apply a jaw force within an appropriate jaw force range to tissue clamped between compliant jaw liner 554 of jaw member 550 and ultrasonic blade 540, and such that, upon receipt of appropriate input, e.g., in a second, opposite rotational direction, into fourth input coupler 194, jaw drive sub-assembly 400 pivots jaw member 550 towards the spaced-apart position to release clamped tissue. Alternatively, jaw drive sub-assembly 400 may be configured to receive separate inputs for opening and closing jaw member 550. In either configuration, jaw drive sub-assembly 400 may be tuned to provide a jaw clamping force, or jaw clamping force within a jaw clamping force range, to tissue clamped between jaw member 550 and ultrasonic blade 540, such as described in U.S. Patent Application Pub. No. 2022/0117622, the entire contents of which are hereby incorporated herein by reference. Alternatively, the jaw drive sub-assembly 400 may include a force limiting feature, e.g., a spring, whereby the clamping force applied to tissue clamped between jaw member 550 and ultrasonic blade 540 is limited to a particular jaw clamping force or a jaw clamping force within a jaw clamping force range, such as described in U.S. Pat. No. 10,368,898, the entire contents of which are hereby incorporated herein by reference.

Actuation assembly 190 is configured to operably interface with a surgical robotic system, e.g., system 10 (FIG. 1), when instrument 110 is mounted on a robotic arm thereof, to enable robotic operation of actuation assembly 190 to provide the above detailed functionality. That is, surgical robotic system 10 (FIG. 1) selectively provides inputs, e.g., rotational inputs to input couplers 191-194 of actuation assembly 190 to articulate end effector assembly 550, clamp tissue between jaw member 550 and ultrasonic blade 540, release tissue (e.g., sealed and/or transected tissue) from between jaw member 550 and ultrasonic blade 540, and, in aspects, rotate end effector assembly 500. However, as noted above, it is also contemplated that actuation assembly 190 be configured to interface with any other suitable surgical systems, e.g., a manual surgical handle, a powered surgical handle, etc.

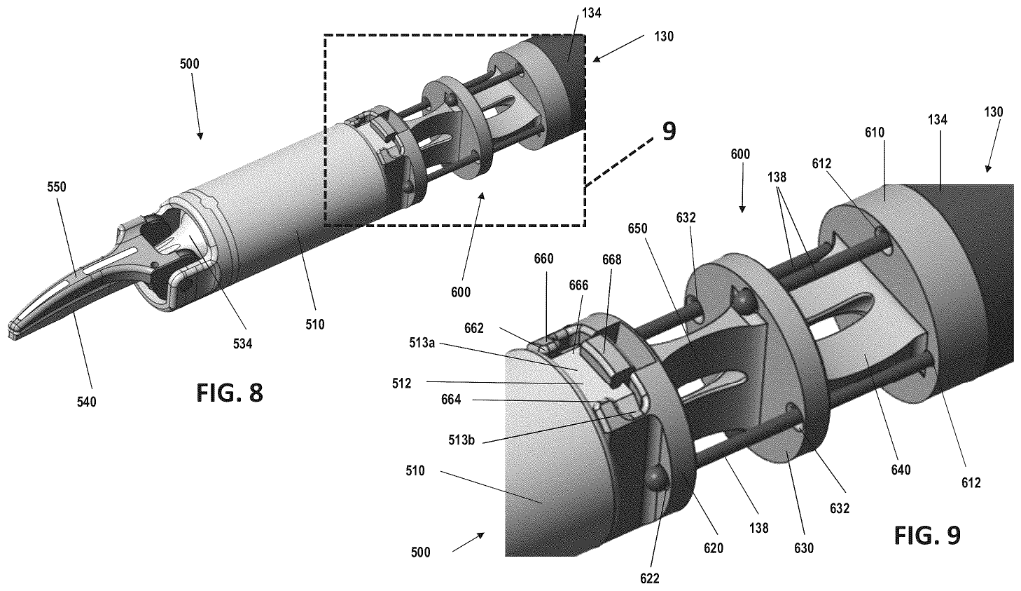

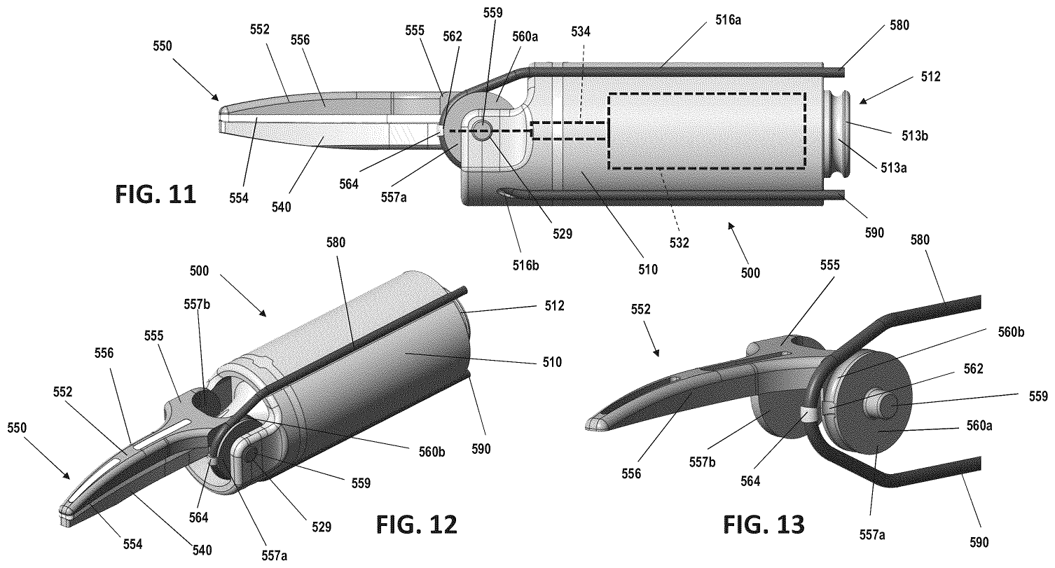

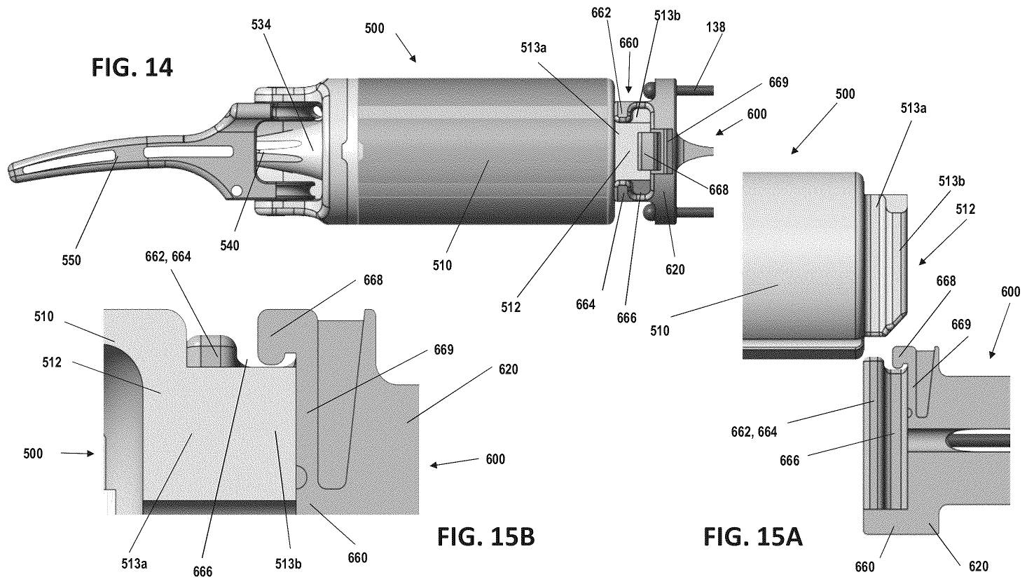

Turing to FIGS. 8-15B, a distal portion of surgical instrument 110 (FIGS. 5-6B) is shown and described in greater detail. As noted above, surgical instrument 110 (FIGS. 5-6B) includes a shaft assembly 130 having a proximal shaft 134 and an articulating section 136 disposed between and interconnecting proximal shaft 134 with end effector assembly 500.

Referring to FIGS. 8 and 9, articulating section 136 of shaft assembly 130 of surgical instrument 110 (FIGS. 5-6B) includes an articulating component 600 formed monolithically from a single piece of material (e.g., a biocompatible polymer or other suitable material), although other configurations are also contemplated. Articulating component 600 includes a proximal disk 610, a distal disk 620, one or more intermediate disks 630 disposed between the proximal and distal disks 610, 620, respectively, and a flexible interconnect 640, 650 interconnecting each pair of adjacent disks 610, 620, 630.

Proximal disk 610 is engaged to a distal end of proximal shaft 134 to thereby secure a proximal end of articulating component 600 to the distal end of proximal shaft 134. Proximal disk 610 defines a plurality of apertures 612 defined longitudinally therethrough. Apertures 612 are radially-spaced about the periphery of disk 610. In aspects, four apertures 612 are provided offset approximately 90 degrees relative to one another, although any other suitable number and/or positioning of apertures 612 is also contemplated. Each aperture 612 is configured to receive one of the articulation cables 138 of articulation sub-assembly 200 of actuation assembly 190 (see FIG. 7) therethrough.

Distal disk 620 is configured to engage and, in aspects, releasably engage, a proximal end of end effector assembly 500 to thereby secure a distal end of articulating component 600 to the proximal end of end effector assembly 500. Distal disk 620 defines a plurality of apertures 622 defined longitudinally therethrough. Apertures 622 are radially-spaced about the periphery of disk 620. In aspects, two apertures 622 are provided offset approximately 180 degrees relative to one another, although any other suitable number and/or positioning of apertures 622 is also contemplated. Each aperture 622 is configured to receive an articulation cable 138 of articulation sub-assembly 200 of actuation assembly 190 (see FIG. 7) therethrough with the articulation cable 138 anchored on a distal side of the aperture 622, e.g., via a ball, knot, ferrule, or other suitable anchor configured to inhibit passage of the distal end of the articulation able 138 proximally through the corresponding aperture 622.

Distal disk 620 further includes a distal connector 660 configured to enable releasable engagement of end effector assembly 500 with articulating component 600, as detailed below, although other suitable configurations including integrated configurations are also contemplated.

The one or more intermediate disks 630 are disposed between the proximal and distal disks 610, 620, respectively. Although detailed below with respect to one intermediate disk 630 (in the singular, for purposes of clarity), it is understood that multiple intermediate disks 630 may be provided. Intermediate disk 630 defines a plurality of apertures 632 longitudinally therethrough. Apertures 632 are radially-spaced about the periphery of disk 630 and are aligned with corresponding apertures 612 of disk 610, e.g., four apertures 632 are provided offset approximately 90 degrees relative to one another. Further, two of apertures 632 are aligned with corresponding apertures 622 of disk 620. In this manner, the articulation cables 138, e.g., four articulation cables 138, extending through apertures 612 of proximal disk 610 also extend through apertures 632 of intermediate disk 630. Two diametrically-opposed articulation cables 138 of the four articulation cables 138 are anchored on distal sides of the corresponding apertures 632 of intermediate disk 630, e.g., via a ball, knot, ferrule, or other suitable anchor configured to inhibit passage of the distal end of the articulation cable 138 proximally through the corresponding aperture 632. The other two diametrically-opposed articulation cables 138 of the four articulation cables 138 extend distally from intermediate disk 630 and through corresponding apertures 622 of distal disk 620 wherein, as noted above, these articulation cables 138 are anchored on the distal side of distal disk 620.

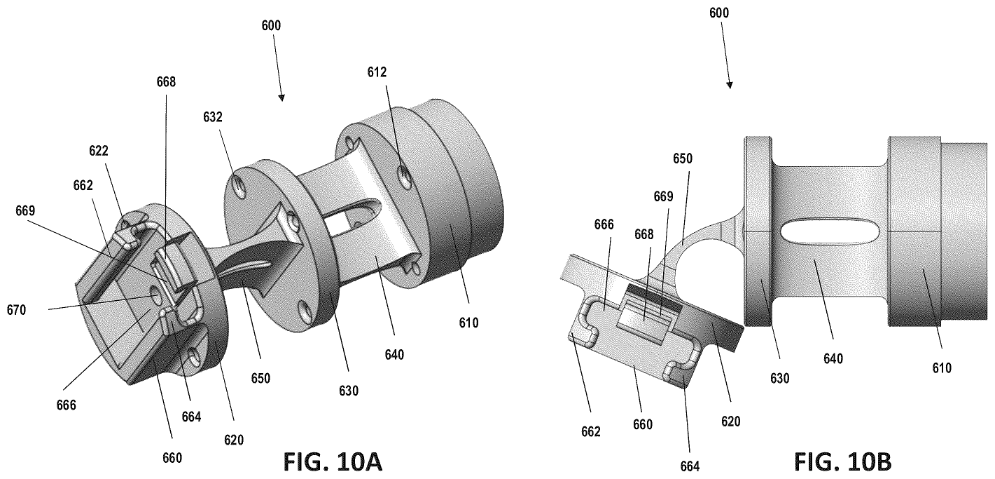

With additional reference to FIGS. 10A and 10B, flexible interconnects 640, 650 interconnect proximal and intermediate disks 610, 630 and intermediate and distal disks 630, 620, respectively. Each flexible interconnect 640, 650 defines a single plane of flexibility. More specifically, flexible interconnects 640, 650 may define beam configurations having a pair of opposed relatively narrow sides and a pair of opposed relatively broad sides. In this manner, each flexible interconnect 640, 650 is configured to flex in directions defined within a single plane extending substantially parallel to the relatively narrow sides and substantially perpendicular to the relatively broad sides. However, other suitable configurations of flexible interconnects 640, 650 each having a single plane of flexibility are also contemplated. Flexible interconnects 640, 650 may define narrowed thickness portions and/or openings defined therethrough to facilitate bending with the respective planes of flexibility thereof. Each flexible interconnect 640, 650 may be configured to flex to a bend angle of at least 60 degrees; in aspects, at least 75 degrees; and in still other aspects, at least 90 degrees. In aspects, flexible interconnects 640, 650 intersect a longitudinal axis defined through proximal shaft 134 and, in some aspects, may be centered on the longitudinal axis defined through proximal shaft 134.

Continuing with reference to FIGS. 8-10B, flexible interconnects 640, 650 are offset approximately 90 degrees relative to one another such that the planes of flexibility defined by flexible interconnects 640, 650 are substantially perpendicular to one another. Further, the two diametrically-opposed articulation cables 138 anchored on the distal side of intermediate disk 630 extend between proximal disk 610 and intermediate disk 630 along a plane that is substantially parallel or coplanar with the plane of flexibility defined by flexible interconnect 640, while the two diametrically-opposed articulation cables 138 anchored on the distal side of distal disk 620 extend between intermediate disk 630 and distal disk 620 along a plane that is substantially parallel or coplanar with the plane of flexibility defined by flexible interconnect 650. In this manner, actuation of the two diametrically-opposed articulation cables 138 anchored on the distal sides of intermediate disk 630 in opposite manners (e.g., tensioning one of the cables 138 and de-tensioning the opposed cable 138) flexes flexible interconnect 640 to bend within the plane of flexibility thereof (with the direction of bending depending upon which of the cable 138 is tensioned and which cable 138 is de-tensioned), while actuation of the two diametrically-opposed articulation cables 138 anchored on the distal sides of distal disk 620 in opposite manners (e.g., tensioning one of the cables 138 and de-tensioning the opposed cable 138) flexes flexible interconnect 650 to bend within the plane of flexibility thereof (with the direction of bending depending upon which of the cable 138 is tensioned and which cable 138 is de-tensioned). In aspects, flexible interconnects 640, 650 are oriented relative to one another and proximal shaft 134 such that flexion of flexible interconnects 640, 650 within the planes of flexibility thereof provides pitch and yaw articulation, respectively, of end effector assembly 500 relative to proximal shaft 134.

With additional reference to FIG. 7, articulation sub-assembly 200 is operably coupled between first and second input couplers 191, 192, respectively, of actuation assembly 190 and the articulation cables 138 (FIG. 9) and configured such that: in response to a rotational input into first coupler 191 in a first direction, articulation sub-assembly 200 actuates the two diametrically-opposed articulation cables 138 anchored on the distal sides of intermediate disk 630 in opposite directions with equal magnitude to articulate end effector assembly 500 in an upward pitch direction; in response to a rotational input into first coupler 191 in a second, opposite direction, articulation sub-assembly 200 actuates the two diametrically-opposed articulation cables 138 anchored on the distal sides of intermediate disk 630 in opposite directions with equal magnitude (oppositely from above) to articulate end effector assembly 500 in an downward pitch direction; in response to a rotational input into second coupler 192 in a first direction, articulation sub-assembly 200 actuates the two diametrically-opposed articulation cables 138 anchored on the distal sides of distal disk 620 in opposite directions with equal magnitude to articulate end effector assembly 500 in an right yaw direction; and in response to a rotational input into second coupler 192 in a second direction, articulation sub-assembly 200 actuates the two diametrically-opposed articulation cables 138 anchored on the distal sides of distal disk 620 in opposite directions with equal magnitude (oppositely from above) to articulate end effector assembly 500 in an left yaw direction. Accordingly, any suitable combination of pitch and/or yaw articulation (or other suitable articulation) can be achieved.

In aspects, articulating component 600 includes one or more lumens such as, for example, a central lumen 670 (FIG. 10A) extending through each of the disks 610, 620, 630 and flexible interconnects 640, 650. Central lumen 670 (FIG. 10A) enables the passage of jaw open and close cables 580, 590, respectively, from proximal shaft 134 through articulating component 600 to end effector assembly 500 to enable pivoting of jaw member 550 towards and away from ultrasonic blade 540, respectively, as detailed below. Electrical wires for delivering electrical signals to drive ultrasonic transducer 532 of end effector assembly 500 may likewise extend through one of the lumens, e.g., central lumen 670 (FIG. 10A); alternatively, electrical communication may be established in any other suitable manner such as, for example, via wires, electrically-conductive structures of surgical instrument 110 (FIGS. 5-7), and/or combinations thereof.

Referring to FIGS. 8 and 11-13, end effector assembly 500, as noted above, may be utilized with surgical instrument 110 (FIGS. 5-7) or any other suitable surgical instrument and generally includes body 510 retaining ultrasonic transducer 532 therein, an ultrasonic horn 534 coupled to and extending distally from ultrasonic transducer 534, an ultrasonic blade 540 coupled to and extending distally from ultrasonic horn 534 and body 510, and jaw member 550 operably coupled to body 510 to enable pivoting of jaw member 550 relative to ultrasonic blade 540 between the spaced-apart position and the approximated position. End effector assembly 500, in addition or as an alternative to the description herein, may include any of the aspects and features of the end effector assembly detailed in U.S. Provisional Patent Application No. 63/325,195, filed on Mar. 30, 2022, the entire contents of which are hereby incorporated herein by reference.

Body 510 of end effector assembly 500 encloses and secures ultrasonic transducer 532 therein. Body 510 includes a proximal connector 512 configured to releasably engage distal connector 660 of articulating component 600 to releasably engage end effector assembly 500 with articulating component 600, although other suitable configurations including integrated configurations are also contemplated. Body 510 further includes first and second cable guide channels 516a, 516b configured to guide jaw open and close cables 580, 590 from jaw member 550 proximally along body 510 to articulating component 600, wherein jaw open and close cables 580, 590 may extend through, about, along or otherwise proximally relative to articulating component 600 and, ultimately, through proximal shaft 134 to connect to jaw drive sub-assembly 400 of actuation assembly 190 (see FIG. 7). Body 510 may also include proximal apertures (not shown) for electrical wires and/or other suitable electrically-conductive pass-through connectors to enable passage of electrical signals through body 510 and to ultrasonic transducer 532 to drive ultrasonic transducer 532. Body 510, in aspects, may be configured to engage ultrasonic horn 534 and sealingly enclose ultrasonic transducer 532 therein similarly as detailed in Patent Application Publication Nos. US 2019/0231385, US 2021/0369295, and/or WO 2021/006984, the entire contents of each of which is hereby incorporated herein by reference.

Continuing with reference to FIGS. 8 and 11-13, ultrasonic transducer 532 may include a stack of piezoelectric elements secured, under pre-compression between proximal and distal end masses or a proximal end mass and ultrasonic horn 534 with electrodes (not shown) electrically coupled between piezoelectric elements of the stack of piezoelectric elements to enable energization thereof to produce ultrasonic energy. However, other suitable ultrasonic transducer configurations, including plural transducers and/or non-linear transducers are also contemplated. Electrical lead wires or other suitable electrical communication paths (not shown) are configured to connect the electrodes of ultrasonic transducer 532 with an ultrasonic generator (not shown) to enable an electrical drive signal generated by the ultrasonic generator to be imparted to the stack of piezoelectric elements of ultrasonic transducer 532 to energize the stack of piezoelectric elements to produce ultrasonic energy for transmission to ultrasonic blade 540 via ultrasonic horn 534 to treat tissue, e.g., seal, transect, dissect, score, perform an otomy, or otherwise treat tissue.

Ultrasonic horn 534 is engaged to the stack of piezoelectric elements of ultrasonic transducer 532 and extends distally therefrom. Ultrasonic blade 540 extends distally from ultrasonic horn 534 and distally from body 510. Ultrasonic blade 540 may define a curved configuration wherein the directions of movement of jaw member 550 between the spaced-apart and approximated positions are perpendicular to the direction of curvature of ultrasonic blade 540. However, it is also contemplated that ultrasonic blade 540 define a straight configuration or that ultrasonic blade 540 additionally or alternatively curve towards or away from jaw member 550; that is, where the directions of movement of jaw member 550 between the spaced-apart and approximated positions are coplanar or parallel to the direction of curvature of ultrasonic blade 540. Multiple curvatures of ultrasonic blade 540 (in the same or different directions) and/or combinations of curved and linear portions of ultrasonic blade 540 are also contemplated. Likewise, some portions or surfaces of ultrasonic blade 540 may be curved while others are not curved. Ultrasonic blade 540 may additionally or alternatively taper in width (a dimension perpendicular to the directions of movement of jaw member 550 in a proximal-to-distal direction and/or in height (a dimension parallel or coplanar with the directions of movement of jaw member 550) in a proximal-to-distal direction. Other configurations are also contemplated.

Jaw member 550 of end effector assembly 500, as noted above, includes rigid structural frame 552 and compliant jaw liner 554. Rigid structural frame 552 includes a bifurcated proximal portion 555 (e.g., to receive ultrasonic blade 540 therebetween) and an elongated distal portion 556 extending distally from bifurcated proximal portion 555. Bifurcated proximal portion 555 includes first and second spaced-apart jaw flags 557a, 557b. Pivot bosses 559 (only one of which is shown) are aligned with one another (thereby defining a pivot axis), extend outwardly from flags 557a, 557b, and are configured for receipt within opposing apertures 529 (only one of which is shown) of body 510 to thereby pivotably couple jaw member 550 with body 510. One of the jaw flags, e.g., jaw flag 557a, further defines a pulley 560a including an annular channel 560b defined about an outer periphery thereof. A notch 562 is defined within pulley 560a in communication with annular channel 560b.

Jaw open and close cables 580, 590, respectively, may be formed via a single cable having a looped distal end disposed between jaw open cable 580 and jaw close cable 590. More specifically, as shown in FIG. 13, the looped distal end of the single cable defining jaw open and close cables 580, 590, respectively, may be disposed about pulley 560a, at least partially seated within annular channel 560b of jaw member 550. A keying collar 564 fixed, e.g., as a crimp or in any other suitable manner, about the looped distal end of the single cable defining jaw open and close cables 580, 590, respectively, is fixed (permanently or removably) within notch 562, e.g., via welding, adhesion, or mechanical engagement, to thereby define an anchor point where the looped distal end of the single cable defining jaw open and close cables 580, 590, respectively, is fixed relative to pulley 560a. Thus, when jaw open cable 580 is pulled proximally (and jaw close cable 590 is de-tensioned), jaw member 550 is urged to pivot away from ultrasonic blade 540 towards the open position. On the other hand, when jaw close cable 590 is pulled proximally (and jaw open cable 580 is de-tensioned), jaw member 550 is urged to pivot towards ultrasonic blade 540 towards the closed position.

With additional reference to FIG. 7, jaw open and close cables 580, 590, respectively, extend proximally through first and second cable guide channels 516a, 516b, respectively, through and/or about articulating component 600 (FIG. 9), and through proximal shaft 134 to connect with jaw drive sub-assembly 400 of actuation assembly 190. More specifically, jaw open and close cables 580, 590, respectively, may be coupled to jaw drive sub-assembly 400 in an equal and opposite manner such that a rotational input in a first direction to an input coupler 194 associated with actuation assembly 190 (FIG. 7) actuates jaw drive sub-assembly 400 to pull jaw open cable 580 proximally while de-tensioning jaw close cable 590 (allowing jaw close cable 590 to move distally), thereby pivoting jaw member 550 away from ultrasonic blade 540 towards the open position, and such that a rotational input in a second, opposite direction to input coupler 194 actuates jaw drive sub-assembly 400 to pull jaw close cable 590 proximally while de-tensioning jaw open cable 580 (allowing jaw open cable 580 to move distally), thereby pivoting jaw member 550 towards ultrasonic blade 540 towards the closed position, e.g., to grasp tissue between jaw member 550 and ultrasonic blade 540.

Turning to FIGS. 10A, 11, and 14-15B, as noted above, in aspects, body 510 of end effector assembly 500 includes a proximal connector 512 configured to releasably engage distal connector 660 of distal disk 620 of articulating component 600 to releasably engage end effector assembly 500 with articulating component 600 and, thus, the remainder of surgical instrument 110 (FIGS. 5-8). Further, it is understood that the features of connector 660 and proximal connector 512 detailed below may be reversed.

Connector 660 includes a pair of spaced-apart rails 662, 664 defining a slot 666 therebetween. Connector 660 may further include a spring tab 668 defined at the free end of a resilient living hinge 669 disposed towards the open end of slot 666 of connector 660.

Proximal connector 512 includes a proximally-extending neck 513a and a head 513b disposed at the free end of neck 513a. Head 513b defines a width (or diameter) greater than a width (or diameter) of neck 513a. More specifically, head 513b is configured to slide transversely between rails 662, 664 and into slot 666 of connector 660 but is sufficiently dimensioned to inhibit longitudinal passage between rails 662, 664 and out of slot 666. Neck 513a, on the other hand, is configured to extend longitudinally between and distally from rails 662, 664 when head 513b is engaged within slot 666. With head 513b fully received within slot 666, spring tab 668 is configured to engage head 513b to thereby secure head 513b within slot 666 transversely between the closed end of slot 666 and spring tab 668, thereby securely engaging end effector assembly 500 with articulating component 600. In aspects, end effector assembly 500 is permitted to rotate relative to articulating component 600 despite this secure engagement; in other aspects, end effector assembly 500 is rotationally fixed. Spring tab 668 may be manually displaced, against the bias thereof, to disengage spring tab 668 from head 513b and enable transverse withdrawal of proximal connector 512 from slot 666 of connector 660, thereby disengaging end effector assembly 500 from articulating component 600.

Proximal connector 512 and connector 660 may define cooperating electrical contacts (not explicitly shown) that are configured to electrically couple with one another upon engagement of end effector assembly 500 with articulating component 600 to thereby connect ultrasonic transducer 532 of end effector assembly 500 to electrical wires and/or structures extending through the remainder of surgical instrument 110 (FIGS. 5-7) and ultimately connected to the ultrasonic generator, thus enabling driving of ultrasonic transducer 532 when end effector assembly 500 is engaged with articulating component 600. Alternatively, manual electrical connections may be established.

Jaw open and close cables 580, 590, respectively, may additionally or alternatively be releasably engagable with jaw member 550 to facilitate releasable operable engagement of end effector assembly 500 with articulating component 600. More specifically, jaw open and close cables 580, 590, respectively, may be releasably seated within annular channel 560b of pulley 560a of jaw member 550 via releasable engagement of keying collar 564 within notch 562. Other suitable releasable engagements are also contemplated such as, for example, intermediate connectors along jaw open and close cables 580, 590, respectively, to enable releasable engagement between proximal and distal portions of jaw open and close cables 580, 590, respectively.

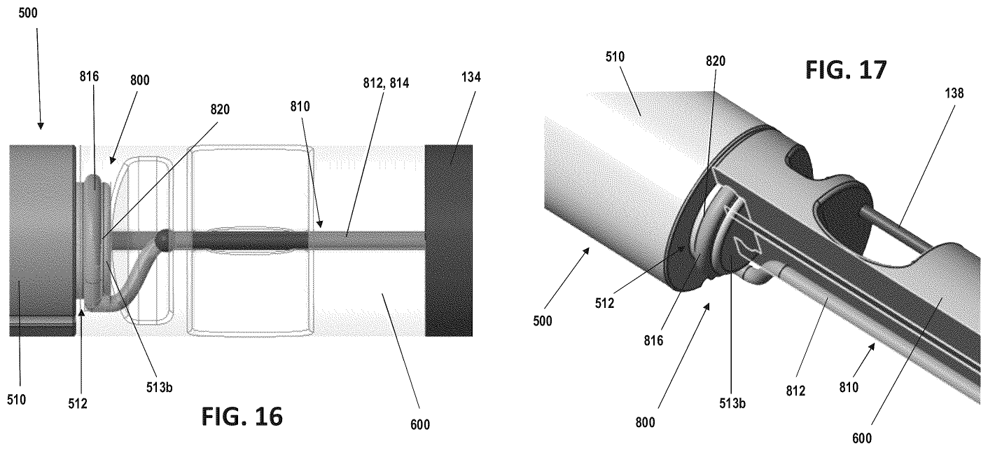

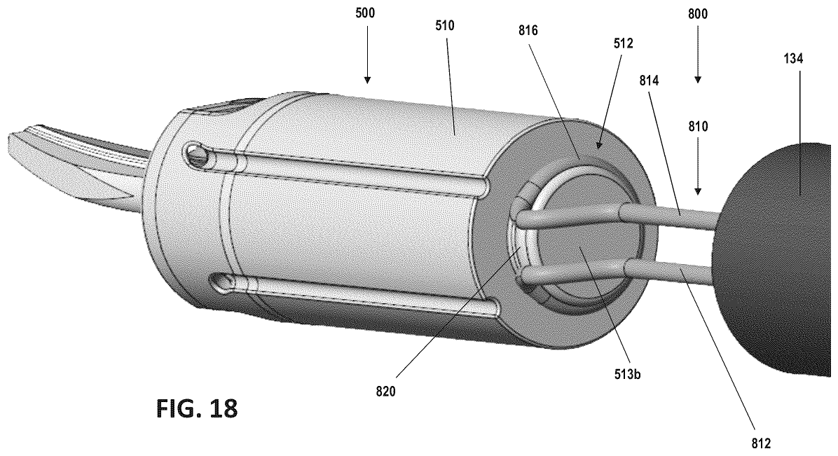

With reference to FIGS. 16-18, a rotation mechanism configured for use with surgical instrument 110 (FIGS. 5-7) or any other suitable surgical instrument is shown generally identified by reference numeral 800. Rotation mechanism 800 includes a cable 810 having a clockwise rotation cable portion 812, a counterclockwise rotation cable portion 814, and a partially looped cable portion 816. Clockwise and counterclockwise rotation cable portions 812, 814, respectively, of cable 810 extend proximally through and/or about articulating component 600 and through proximal shaft 134 to connect with actuation assembly 190. More specifically, Clockwise and counterclockwise rotation cable portions 812, 814, respectively, of cable 810 may be coupled to a rotation drive assembly (not explicitly shown) in an equal and opposite manner such that a suitable rotational input, e.g., in a first direction, to an input coupler 193 associated with actuation assembly 190 (FIG. 7) pulls clockwise rotation cable portion 812 proximally while de-tensioning and allowing counterclockwise rotation cable portion 814 to move distally and such that a suitable rotational input, e.g., in a second, opposite direction, to input coupler 193 de-tensions clockwise rotation cable portion 812 allowing clockwise rotation cable portion 812 to move distally and pulls counterclockwise rotation cable portion 814 proximally. Alternatively, actuation assembly 190 (FIG. 7) may be configured to receive separate inputs for proximally pulling clockwise and counterclockwise rotation cable portions 812, 814, respectively.

Rotation mechanism 800 further includes proximal connector 512 of end effector assembly 500 which, as noted above, includes a head 513b coupled to articulating component 600 (FIGS. 8-9) or other suitable portion of surgical instrument 110 (FIG. 7), e.g., via removable or integral engagement. More specifically, in aspects, whether removable or integral, head 513b is rotatably coupled to articulating component 600 (FIGS. 8-9) or other suitable portion of surgical instrument 110 (FIG. 7). Head 513b defines a disk-shaped configuration (facilitating the above-noted rotatable engagement) and includes an annular track 820 defined about the annular perimeter of the disk-shaped head 513b. Partially looped cable portion 816 extends circumferentially greater than 180 degrees but less than 360 degrees and is seated within annular track 820 of head 513b of proximal connector 512. As a result of this configuration, when clockwise rotation cable portion 812 is pulled proximally (and counterclockwise rotation cable portion 814 moves distally), cable 810 slides through annular track 820 in a clockwise direction. As cable 810 slides through annular track 820, friction between cable 810 and annular track 820 generates a clockwise torque at proximal connector 512, thereby urging end effector assembly 500 to rotate in a clockwise direction about and relative to articulating component 600 and proximal shaft 134. On the other hand, when counterclockwise rotation cable portion 814 is pulled proximally (and clockwise rotation cable portion 812 moves distally), cable 810 slides through annular track 820 in a counterclockwise direction. As cable 810 slides through annular track 820, friction between cable 810 and annular track 820 generates a counterclockwise torque at proximal connector 512, thereby urging end effector assembly 500 to rotate in a counterclockwise direction about and relative to articulating component 600 and proximal shaft 134. Suitable structures such as, for example, roughened surfaces, protrusions, indents, teeth, and the like may be provided on cable 810 and/or annular track 820 to increase friction and, thus, increase torque generation and/or to otherwise facilitate rotation or rotational control of end effector assembly 500 about and relative to articulating component 600 and proximal shaft 134.

Thus, as detailed above, rotation mechanism 800 enables selective rotation of end effector assembly 500 relative to articulating component 600 and proximal shaft 134, e.g., in response to a corresponding input to input coupler 193 of actuation assembly 190 (FIG. 7). This rotation may be provided in addition to the multi-plane, e.g., pitch and yaw, articulation provided by articulating component 600, as detailed above; may be in addition to single-plane (e.g., pitch or yaw) articulation provided by articulating component 600; or may be utilized with (or without) any other suitable articulation mechanism.

Although this disclosure is detailed with respect to an ultrasonic surgical instrument including a distally positioned transducer, this disclosure is equally applicable to other suitable surgical instruments. For example and without limitation, the transducer, ultrasonic horn, and ultrasonic blade may be replaced with energy-generating electrodes and an opposing jaw member configured for positioning opposite the movable jaw member. In such configurations, the proximal body of the end effector assembly may house power, energy-generating, and/or control electronics to operate an energy-based component associated with the fixed jaw member and/or the movable jaw member, e.g., either or both including an RF electrode for monopolar or bipolar tissue treatment, a thermal cutting element configured to thermally treat tissue, a microwave probe, combinations of various different energy modalities, etc.

It will be understood that various modifications may be made to the aspects and features disclosed herein. Therefore, the above description should not be construed as limiting, but merely as exemplifications of various configurations. Those skilled in the art will envision other modifications within the scope and spirit of the claims appended hereto.

What is claimed is:

1. An articulating surgical instrument, comprising:
a housing;
a shaft extending distally from the housing;
an end effector assembly; and
an articulating component interconnecting the shaft and end effector assembly and including:
a proximal disk connected to the shaft;
a distal disk connected to the end effector assembly;
an intermediate disk;
a first flexible interconnect connecting the proximal and intermediate disks; and
a second flexible interconnect connecting the intermediate and distal disks,
wherein the first flexible interconnect defines a single plane of bending to enable articulation of the end effector assembly relative to the shaft within a first plane, and
wherein the second flexible interconnect defines a single plane of bending substantially perpendicular to the single plane of bending of the first flexible interconnect to enable articulation of the end effector assembly relative to the shaft within a second plane substantially perpendicular to the first plane.

2. The articulating surgical instrument according to claim 1, wherein the articulating component is a monolithic, single piece of material.

3. The articulating surgical instrument according to claim 1, wherein each of the first and second flexible interconnects defines a beam configuration having a pair of opposed relatively narrow sides and a pair of opposed relatively broad sides.

4. The articulating surgical instrument according to claim 3, wherein each of the first and second flexible interconnects is aligned relative to a longitudinal axis defined through the shaft.

5. The articulating surgical instrument according to claim 1, further comprising a first pair of opposed articulation cables extending from the shaft through the proximal and intermediate disks, the first pair of opposed articulation cables anchored distally of the intermediate disk and configured to move in opposite directions to articulate the end effector assembly relative to the shaft within the first plane.

6. The articulating surgical instrument according to claim 5, further comprising a second pair of opposed articulation cables extending from the shaft through the proximal, intermediate, and distal disks, the second pair of opposed articulation cables anchored distally of the distal disk and configured to move in opposite directions to articulate the end effector assembly relative to the shaft within the second plane.

7. The articulating surgical instrument according to claim 6, wherein the first and second pairs of opposed articulation cables are offset about 90 degrees relative to one another.

8. The articulating surgical instrument according to claim 1, wherein the end effector assembly includes:
a body;
an ultrasonic transducer housed within the body; and
an ultrasonic blade extending distally from the body and configured to treat tissue with ultrasonic energy produced by the ultrasonic transducer.

9. The articulating surgical instrument according to claim 8, wherein the end effector assembly further includes a jaw member pivotable relative to the ultrasonic blade between an open position and a closed position for clamping tissue therebetween.

10. The articulating surgical instrument according to claim 9, further comprising at least one jaw actuation cable routed through the articulating component to the end effector assembly.

11. The articulating surgical instrument according to claim 1, wherein the distal disk is connected to the end effector assembly by a releasable engagement.

12. The articulating surgical instrument according to claim 11, wherein the releasable engagement includes:
a first connector; and
a second connector including first and second rails defining a slot therebetween and an engagement tab positioned towards an open end of the slot,
wherein the first connector is transversely slidable between the first and second rails and into the slot, and wherein the engagement tab is configured to releasably engage the first connector within the slot upon sufficient transverse sliding of the first connector into the slot.

13. An articulating surgical instrument, comprising:
a housing;
a shaft assembly extending distally from the housing, the shaft assembling including a proximal shaft and a distal articulating section; and
an end effector assembly releasably coupled to the distal articulating section of the shaft assembly by a releasable engagement including:
a first connector disposed on one of the end effector assembly or the distal articulating section; and
a second connector disposed on another of the end effector assembly or the distal articulating section and including first and second rails defining a slot therebetween and an engagement tab positioned towards an open end of the slot,
wherein the first connector is transversely slidable between the first and second rails and into the slot, and wherein the engagement tab is configured to releasably engage the first connector within the slot upon sufficient transverse sliding of the first connector into the slot.

14. The articulating surgical instrument according to claim 13, wherein the first connector is disposed on the end effector assembly and wherein the second connector is disposed on the distal articulating section.

15. The articulating surgical instrument according to claim 13, wherein the second connector further including a living hinge, wherein the engagement tab is disposed at a free end of the living hinge.

16. The articulating surgical instrument according to claim 13, wherein the end effector assembly includes:
a body having the first or second connector extending therefrom;
an ultrasonic transducer housed within the body; and
an ultrasonic blade extending distally from the body and configured to treat tissue with ultrasonic energy produced by the ultrasonic transducer.

17. The articulating surgical instrument according to claim 16, wherein the end effector assembly further includes a jaw member pivotable relative to the ultrasonic blade between an open position and a closed position for clamping tissue therebetween.

18. A rotating and articulating surgical instrument, comprising:
a housing;
a shaft assembly extending distally from the housing, the shaft assembling including a proximal shaft and a distal articulating section;
an end effector assembly coupled to the distal articulating section, wherein the distal articulating section is configured to articulate the end effector assembly relative to the proximal shaft within at least one plane, the end effector assembly including a proximal head defining an annular track; and
a rotation mechanism including a cable extending through the distal articulating section of the shaft assembly to the end effector assembly, the cable including a first portion, a second portion, and a partially looped portion interconnecting the first and second portions, the partially looped portion disposed at least partially within the annular track of the proximal head of the end effector assembly,
wherein movement of the first and second portions of the cable in opposite directions slides the partially looped portion through the annular track to generate torque to thereby rotate the end effector assembly relative to the shaft assembly.

19. The rotating and articulating surgical instrument according to claim 18, wherein movement of the first and second portions of the cable in opposite directions in a first manner slides the partially looped portion through the annular track in a clockwise direction to generate torque to thereby rotate the end effector assembly relative to the shaft assembly in the clockwise direction, and wherein movement of the first and second portions of the cable in opposite directions in a second, opposite manner slides the partially looped portion through the annular track in a counterclockwise direction to generate torque to thereby rotate the end effector assembly relative to the shaft assembly in the counterclockwise direction.

20. The rotating and articulating surgical instrument according to claim 18, wherein movement of the first and second portions of the cable in opposite directions slides the partially looped portion through the annular track and, as a result of friction between the partially looped portion of the cable and the annular track, torque is generated to thereby rotate the end effector assembly relative to the shaft assembly.
